(19) United States
(12) Patent Application Publication
(10) Pub. No.: US 2011/0021982 A1
(43) Pub. Date: Jan. 27, 2011

(54) DISPENSING DEVICE WITH BYPASS

(75) Inventor: Wilhelm A. Keller, Merlischachen (CH)

Correspondence Address: FOLEY AND LARDNER LLP;SUITE 500, 3000 K STREET NW, WASHINGTON, DC, 20007, US

(73) Assignee: Medmix Systems AG

(21) Appl. No.: 12/933,854

(22) PCT Filed: Mar. 20, 2009
(86) PCT No.: PCT/CH09/00099

§ 371 Date: Sep. 21, 2010

(30) Foreign Application Priority Data
Mar. 25, 2008 (CH) 432/08

(51) Int. Cl. A61M 5/31

(52) U.S. Cl. 604/82; 604/191

(57) ABSTRACT

A device for dispensing multiple components has a syringe housing comprising at least one storage container that is divided into at least two chambers and has a bypass arrangement, and a second storage container with or without a bypass arrangement, the syringe housing being realized as part of a double syringe or double cartridge having a double plunger and a common outlet. The bypass arrangement comprises at least two indentations. The device is simple to manufacture and provides for an improved mixing of components, in particular powder and a fluid, and improves dispensing under hygienic conditions.

The present invention relates to a device for dispensing multiple components, comprising at least one storage container that is divided into at least two chambers and has a bypass arrangement and that is a part of a syringe housing of a double syringe.

In medicine it is known practice to store adhesives, bone cements, or gels as two-component materials in double syringes or double cartridges. To apply them, the materials are mixed by means of a static mixer or a spray system or the like, and dispensed.

Mainly in medicine, materials that are composed of three or four components are increasingly being used. These are separately stored in individual receptacles and according to a prior art, the third or fourth component, respectively, is brought together with the corresponding liquid and, if required, mixed or dissolved and then filled into a double syringe in order to be subsequently mixed during the dispensing operation. All in all, this is a complicated procedure, particularly in view of the required compliance to the hygienic regulations.

A device according to the introduction is known from EP-0 219 160 B2, where each storage container has as a bypass arrangement one respective indentation at the inner wall surface, which shows as a salient part at the external surface of the wall. Since there is only one indentation, resp. corresponding salient part at each container, its dimension must be relatively great, which can cause production problems. Further, the storage containers have different diameters, and each container has a forward piston as well as a floating rearward piston.

In the case of dissimilar substances, particularly if they are in powder form and liquid, respectively, a quick and thorough mixture is decisive for their effective application, and it is on the background of this prior art the object of the present invention to provide for a device that is relatively simple and inexpensive to manufacture and which ensures a quick and effective mixture of the different substances. This object is attained by the device according to claim 1. Further exemplary embodiments and advantages are defined in the dependent claims.

The invention will be explained in more detail hereinafter with reference to drawings of exemplary embodiments.

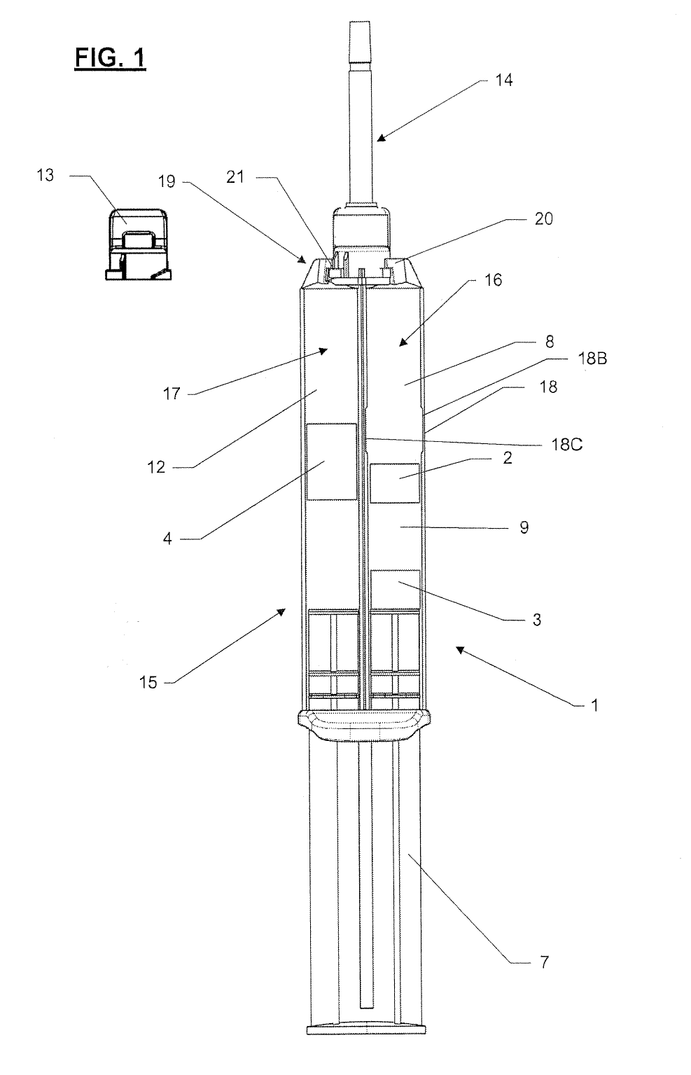

FIG. 1 shows a first exemplary embodiment of a device according to the invention in a longitudinal partial section,

FIGS. 2-4 show three different steps during the mixture of two components and the subsequent dispensing of the mixture of three components,

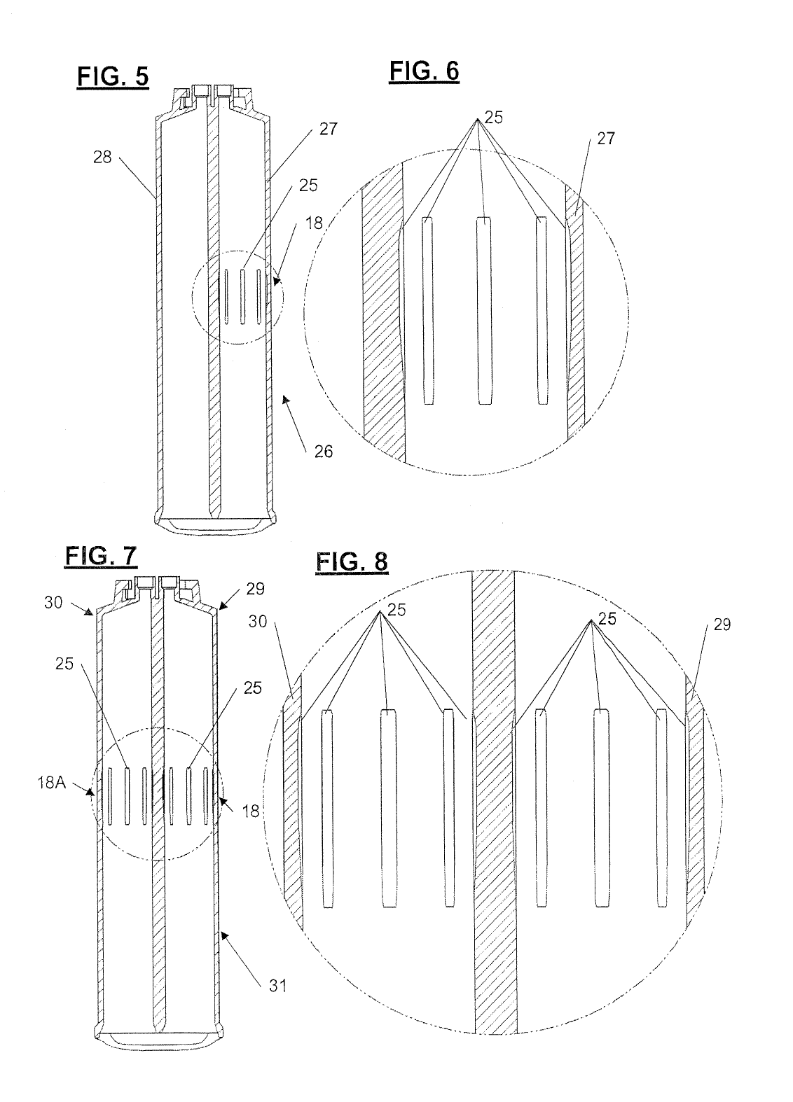

FIG. 5 shows a variant of the bypass arrangement of the device of FIG. 1,

FIG. 6 shows an enlarged detail of FIG. 5,

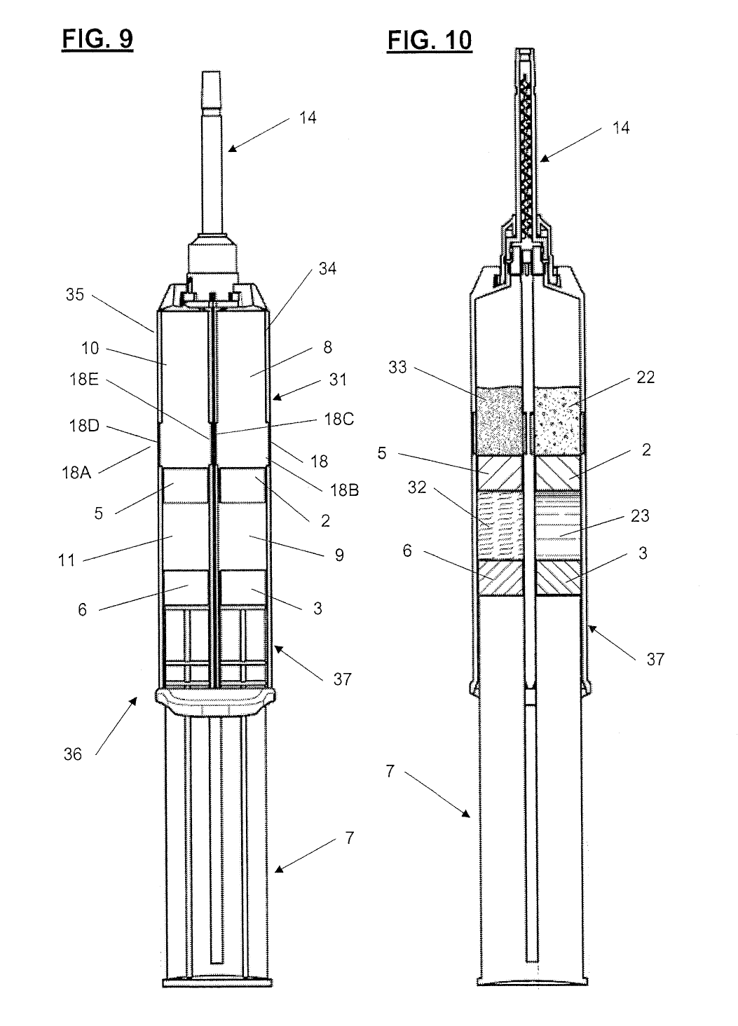

FIG. 7 shows a variant of the bypass arrangement of the device of FIG. 9,

FIG. 8 shows an enlarged detail of FIG. 7,

FIG. 9 shows a second embodiment of a device according to the invention in the empty condition,

FIG. 10 shows the device of FIG. 9 in a longitudinal section and filled.

The partial section of FIG. 1 shows device 1 with a syringe housing 15 having storage containers 16 and 17, as well as a mixer 14 and double plunger 7. Storage container 16 has a bypass arrangement 18 as well as two pistons 2 and 3. In the first embodiment of the invention the bypass arrangement 18 comprises at least two indentations 18B and 18C within one container with no corresponding salient parts because being of a smaller dimension each than the single indentation of the prior art. The other storage container 17 has one piston 4. Outlet flange 19 of the syringe housing is provided with coupling means 20, here in the form of bayonet coupling means. These bayonet coupling means 20 cooperate with bayonet coupling means 21 on mixer 14. For the purposes of the present invention, other coupling means may also be provided, and instead of the mixer, a spray system that is known in the art per se or other accessories may be provided.

During transport, the filled syringe is closed by a closure cap 13. The pistons divide the storage containers into different chambers so that a forward chamber 8 and a rearward chamber 9, with respect to the outlet, are created in storage container 16, and a chamber 12 in storage container 17.

Referring to FIGS. 2 to 4, the operations of mixing resp. dissolving and of jointly dispensing all components are explained in more detail. In FIG. 2, forward chamber 8 is filled with a powder 22, rearward chamber 9 with a liquid 23, and chamber 12 with another liquid, gel, or paste 24. FIG. 2 illustrates the starting position in which forward piston 2 is located behind the bypass arrangement and thus no liquid may exit. In this position, the closure cap is attached to the double syringe.

In the position of FIG. 3, closure cap 13 has been removed and mixer 14 attached, and double plunger 7 has been moved toward the outlet, thereby pushing rearward piston 3 and thus also forward piston 2 forward until the latter is located at the height of bypass arrangement 18. In this position, liquid 23 starts flowing forward into powder 22 to dissolve it.

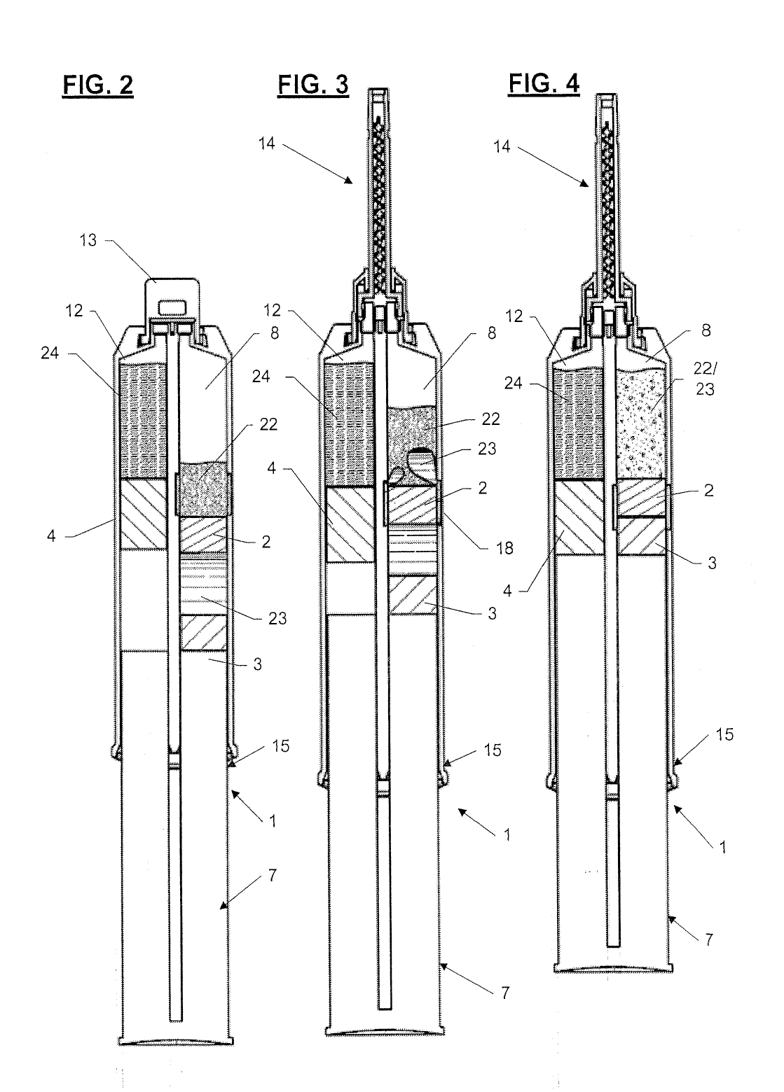

In the position of FIG. 4, all of the liquid has been transferred to the powder to form a solution 22/23 therein, while bypass arrangement 18 is again closed by piston 2 and thus no material may flow back. In the position of FIG. 4, double plunger 7 starts actuating piston 4 too, so that both media reach mixer 14 simultaneously to be mixed therein and subsequently dispensed.

In FIGS. 5 and 6, a preferred embodiment variant of bypass arrangement 18 is illustrated. In the first exemplary embodiment according to FIGS. 1 to 4, bypass arrangement 18 is in the form of two indentations 18B and 18C at the inner wall of the container, whereas in a preferred embodiment variant according to FIGS. 5 and 6, the bypass arrangement 18 is in the form of multiple elongated recesses 25. These recesses 25 are located on the inside of storage cylinder 27 of syringe housing 26 while storage container 28 may be the same as in the previous example. The remaining elements are the same as in the previous example, and so are the two pistons 2 and 3, or single piston 4, respectively.

The design of the bypass arrangement in the form of multiple elongated recesses offers various advantages. Thus, on one hand, the mixture of the liquid in chamber 9 and of the powder in chamber 8 is substantially improved, and it is simpler with regard to the manufacturing technique to produce a number of small recesses than a larger indentation and salient part as it is described in the prior art. This is particularly true for syringes having a small internal diameter. The number of recesses may vary depending on the intended use.

In FIGS. 7 and 8, a second embodiment variant is illustrated in which both storage containers 29 and 30 of syringe housing 31 have respective bypass arrangements 18 and 18A, each bypass arrangement consisting of a number of recesses 25. Although it is advisable from the point of view of the manufacturing technique to design the two bypass arrangements or the individual recesses of the bypasses in the same dimensions in both storage containers, this is not absolutely necessary, and they may also be different from one container to another, depending on the kind of the substances that are to be mixed. Furthermore, both the length and the location of the recesses may vary between the containers and between themselves.

FIGS. 9 and 10 illustrate an exemplary embodiment where both storage containers 34 and 35 of syringe housing 37 of device 36 are provided with respective bypass arrangements 18 and 18A, with indentations 18B, 18C; 18D, 18E. In this exemplary embodiment, storage container 34 is the same as storage container 16 of the previous example and has the same chambers 8 and 9 as well as pistons 2 and 3, and double plunger 7 is also the same as in the previous examples. Analogously, the other storage container 35 has a forward chamber 10 as well as a rearward chamber 11, and the two pistons 5 and 6. In the view of FIG. 10, both storage containers are filled, the rearward chambers each containing a liquid 23 and the forward chamber a powder 24, a liquid, gel, or paste. The operation of this double syringe is the same as described in the previous example. Instead of indentations 18B and 18C a number of recesses 25 can be chosen as bypass arrangement.

The containers of a syringe or cartridge can have the same diameter or different diameters, with a proportion of for example up to 1:10.

1. A device for dispensing multiple components, comprising:
at least one storage container, divided into at least two chambers, including a bypass arrangement,
wherein the at least one storage container is a part of a syringe housing of a double syringe or double cartridge having a double plunger and a common outlet flange
wherein the bypass arrangement has includes at least two indentations or recesses.

2. The device of claim 1, wherein the syringe housing includes one storage container comprising a bypass arrangement and two pistons and one storage container comprising one piston.

3. The device of claim 1, wherein the syringe housing includes two storage containers, wherein each of the storage containers comprises a bypass arrangement and two pistons.

4. The device of claim 1, wherein the common outlet includes a mixer, a spray system, or another accessory.

5. The device of claim 1, wherein the flange of the syringe housing includes coupling elements for a bayonet, push-on, or snap coupling.

6. The device of claim 1, wherein the recesses of the bypass arrangement, for each storage container, have different lengths and/or positions.
